(19) United States
(12) Patent Application Publication
(10) Pub. No.: US 2026/0234515 A1
(43) Pub. Date: Aug. 13, 2026

(54) PROCESS FOR MANUFACTURING SOLID PARTICLES COMPRISING AT LEAST ONE AMINOCARBOXYLATE COMPLEXING AGENT

(71) Applicant: BASF SE, Ludwigshafen am Rhein (DE)

(72) Inventors: Matthias ARNDT, Ludwigshafen Am Rhein (DE); Michael Klemens MUELLER, Ludwigshafen am Rhein (DE); Markus HARTMANN, Ludwigshafen am Rhein (DE); Matthias VOGES, Muenster (DE); Frank JAEKEL, Ludwigshafen am Rhein (DE); Lothar SEIDEMANN, Ludwigshafen am Rhein (DE)

(21) Appl. No.: 19/475,281

(22) PCT Filed: Apr. 3, 2024
(86) PCT No.: PCT/EP2024/059090

§ 371 Date: Oct. 14, 2025

(30) Foreign Application Priority Data
Apr. 14, 2023 (EP) 23167896.2

(51) Int. Cl. C11D 17/06; C11D 1/722; C11D 3/04; C11D 3/20; C11D 3/33; C11D 3/36; C11D 3/37; C11D 3/386; C11D 3/39

(52) U.S. Cl. CPC C11D 17/06; C11D 1/722; C11D 3/046; C11D 3/2034; C11D 3/2082; C11D 3/2093; C11D 3/33; C11D 3/36; C11D 3/3723; C11D 3/38609; C11D 3/3917; C11D 2111/14

(57) ABSTRACT

The present invention relates to a process for manufacturing solid particles comprising at least one aminocarboxylate complexing agent.

The present invention relates to a process for manufacturing solid particles comprising at least one aminocarboxylate complexing agent.

The inventive solid particles are solid and may be in the form of a powder or a granule. The inventive solid particles are preferably free-flowing.

Granules and powders have the advantage of being essentially water-free. That means that in case of shipping, no extra water has to be shipped, and costs for extra weight can be avoided.

To name one example, powders or granules containing typical ingredients of washing or cleaning formulations, e. g. for laundry formulations or dishwashing formulations, are desired by industrial users making and marketing such formulations.

Typical ingredients of washing or cleaning formulations may include builders, surfactants, polymers, inorganic compounds, anionic surfactants, cationic surfactants, non-ionic surfactants and/or chelating agents.

For example, many industrial users prefer to use chelating agents in the form of granules or powders which may contain, in addition to the at least one chelating agent, at least one additional compound, in particular polymers. Some customers also wish to incorporate higher amounts of additional compounds, e. g. polymers, into the granules or powders containing at least one chelating agent.

Methods for providing granules or powders of chelating agents, also in combination with other compounds like polymers, have been described in the art, for example in WO 2015/121170 A1. In general, powders or granules containing one or more compounds may be obtained, for example, by spray-drying or spray-granulation.

US 2023/0025816 A1 describes a process for making a granule comprising (inter alia) MGDA or IDS, wherein water is removed by spray-granulation in a fluidized bed, and wherein the resultant granule is subsequently treated with air or an inert gas. The publication does not disclose a spray-drying process, and neither the degree of crystallinity of the resulting granules.

However, solid particles (e. g. powders or granules) containing aminocarboxylate complexing agents frequently encounter problems. For example, solid particles containing aminocarboxylate complexing agents show a high hygroscopicity and a tendency for coloring, in the presence of peroxy compounds, which are usual components of cleaning agents, just like aminocarboxylate complexing agents. Both properties are undesired.

Thus, there was a need in the art to provide solid particles (e. g. powders or granules) containing aminocarboxylate complexing agents which avoid the problems mentioned above, and to provide a process for manufacturing solid particles (e. g. powders or granules) containing aminocarboxylate complexing agents which leads to solid particles with improved properties (e. g. lower hygroscopicity and/or lower tendency to become colored). Another objective of the present invention was also to provide a process for manufacturing solid particles, preferably free-flowing solid particles, comprising at least one aminocarboxylate complexing agent or its salts with alkali metals, which leads to an increased proportion of crystalline material in the resulting solid particles.

The inventors have surprisingly found that a certain spray-drying process for manufacturing solid (preferably free-flowing) particles comprising at least one aminocarboxylate complexing agent (or its salts with alkali metals) is able to solve the problems set out above.

Thus, one subject of the present invention is a process for manufacturing solid, preferably free-flowing particles comprising at least one aminocarboxylate complexing agent or its salts with alkali metals, by spray-drying a slurry comprising aminocarboxylate complexing agent, in the presence of fine particles under agglomerating conditions.

The term “free-flowing” in the context of the present invention refers to particles which, after storage for 24 hours under humid conditions, (still) do not form lumps. The evaluation may be done visually.

Fine particles refer to particles with an (average) diameter of at most 300 μm. To determine the size of particles, the fines are classified using lab sieves with different mesh sizes.

Agglomerating conditions, in the context of the present invention, may refer to an embodiment wherein particles P1 with a particle diameter which is less than the targeted particle diameter P leave the spray drying apparatus through an exhaust air outlet on the head of the dryer, (ii) are removed by a cyclone and (iii) re-introduced into the drying apparatus, and wherein
particles P1 agglomerate in the spray drying apparatus when they are re-introduced into the spray-drying apparatus, to form particles P2 with an increased average particle diameter and/or weight, as compared to particles P1, and wherein
particles P2, when they have the targeted particle diameter P, fall into the fluidized bed and are, optionally, air-classified and, optionally, post-dried, to receive free-flowing particles P3.

The inventive spray-drying process under agglomerating conditions works in the presence of fine particles (as defined above), which generally leads to a higher degree of crystallinity in the product (since the fine particles work as crystallization seeds), which may be desirable in some applications.

The term “slurry”, as used herein, refers to its common meaning, as known to a person skilled in the art.

(In other words, a “slurry” refers to solid particles distributed in an aqueous phase (wherein the aqueous phase may contain dissolved compounds).)

Further subjects of the present invention are also a solid, preferably free-flowing particle comprising at least one aminocarboxylate complexing agent, obtainable or obtained by the inventive process, and a cleaning agent, preferably dishwashing cleaning agent, comprising an inventive solid, preferably free-flowing particle (preferably obtained or obtainable by the inventive process).

In one embodiment of the present invention, the slurry comprises at least 45% by weight of aminocarboxylate complexing agent and optionally seed particles of solid aminocarboxylate complexing agent.

In another embodiment of the inventive process, the aminocarboxylate complexing agent is selected from the list consisting of MGDA, GLDA, EDDS, IDS, preferably MGDA.

In a further embodiment of the inventive process, the process comprises the steps of
(a) Forming an aqueous slurry, comprising seed particles of solid aminocarboxylate complexing agent, preferably at least 45% by weight of solid aminocarboxylate complexing agent, relative to the total weight of the aqueous slurry,
(b) Spray drying the slurry in a spray drying apparatus, equipped with a fluidized bed,
wherein particles P1 with a particle diameter which is less than the targeted particle diameter P leave the spray drying apparatus through an exhaust air outlet on the head of the dryer, (ii) are removed by a cyclone and (iii) re-introduced into the drying apparatus, and wherein
particles P1 agglomerate in the spray drying apparatus when they are re-introduced into the spray-drying apparatus, to form particles P2 with an increased average particle diameter and/or weight, as compared to particles P1, and wherein
particles P2, when they have the targeted particle diameter P, fall into the fluidized bed and are, optionally, air-classified and, optionally, post-dried, to receive free-flowing particles P3.

Optionally, particles P3 may be compacted into compacted aggregates.

The slurry provided in step (a) may, optionally, contain at least 0.1 weight % of a further organic or inorganic compound or mixtures thereof.

In one embodiment of the present invention, the slurry provided in step (a) contains at least 0.1 weight % of a (co)polymer (B) selected from (co)polymers of (meth)acrylic acid and polyethyleneimines, non-substituted or substituted with alkoxy groups or CH₂COOH groups that may be neutralized with alkali metal, and/or polyaspartates and/or polyepoxysuccinic acid.

In another embodiment of the present invention, the slurry is tempered in step (a) before entering the spray-drying apparatus in step (b). “Tempering” means, in the context of the present invention, keeping the slurry at a temperature of at least 50° C. for a period of 2 minutes to 6 hours.

Preferably, the inventive process is performed in an agglomerative spray-drying apparatus.

In a preferred embodiment, the inventive process is performed in an agglomerative spray-drying apparatus with a height of at least 5 meters, preferably at least 10 meters.

The particles resulting from the inventive process may be amorphous or crystalline.

Optionally, the particles resulting from the inventive process may be matured. The inventive particles lead to good results in the maturing process (also called after-treatment process) of unpublished patent application EP 22192595.1, e. g. according to claim 1 (and the following dependent claims) of this application.
“Maturing” refers, in one embodiment, to a process for preparing a solid, storage stable composition comprising at least one aminocarboxylate complexing agent, comprising the following steps:
a) Providing an initial aminocarboxylate composition with a content of 1 to 70% by weight, preferably 1 to 50% by weight, particularly 1 to 25% by weight solid particles of aminocarboxylates of orthorhombic crystal system, relative to the total initial solid composition,
b) Increasing the relative humidity (rH) to at least 50% and/or adding at least 1% by weight of water or of an aqueous solution (SO) or slurry (SL) with a water content of at least 10% by weight, preferably at least 30% by weight, relative to the total weight of the aqueous solution or slurry,
c) keeping the obtained composition at a temperature of 20 to 95° C. over a period of 1 minute to 5 months,
   drying the resulting solid composition, to a residual moisture content of less than 20%, resulting in a solid aminocarboxylate composition with an increased content of orthorhombic crystal form.

In one embodiment of the present invention the slurry formed in step (a) is heated to a temperature of at least 70° C. (preferably not more than 150° C.) for a period of from 10 minutes to 8 hours (preferably 2 to 4 hours), to increase the content of orthorhombic MGDA in the slurry.

As mentioned above, another subject of the present invention is also a solid, preferably free-flowing particle comprising at least one aminocarboxylate complexing agent. The free-flowing particle may have a particle size of 50 to 1400 μm, preferably 100 to 1000 μm, more preferably 100 to 500, and/or the particle may have a crystallinity of at least 10%, as determined by X-ray diffraction, and/or a content of orthorhombic crystal form of at least 30%, as determined by X-ray diffraction.

The inventive solid, preferably free-flowing particle may have a moisture content of not more than 30 wt. %, preferably less than 20 wt. %, more preferably less than 15 wt. %.

### Applications

A further subject of the present invention is also solid particle, for example a powder or granule, obtained or obtainable according to the inventive process.

Another subject of the present invention is the use of a (solid) particle, e. g. powder or granule, obtained or obtainable according to the inventive process, in laundry or dishwashing applications, preferably dishwashing applications, more preferably, automatic dishwashing applications, or in industrial and institutional cleaning applications.

As one example of a powder or granule, obtained or obtainable according to the inventive process, solid alkali metal salts (A) of an aminocarboxylate complexing agent are mentioned herein.

These inventive powders or granules containing, for example, MGDA, exhibit overall advantageous properties including but not limited to an excellent yellowing behavior, especially in the presence of bleaching agents. They are therefore excellently suitable for the manufacture of cleaning agents that contain at least one bleaching agent, such cleaning agent hereinafter also being referred to as bleach. In particular inventive solid compositions are suitable for the manufacture cleaning agent for fibers or hard surfaces wherein said cleaning agent contains at least one peroxy compound.

Inventive solid compositions (e. g. powders) may easily be converted into compactates and into agglomerates.

Another aspect of the present invention is therefore the use of powder or granule containing a solid alkali metal salt (A) of an aminocarboxylate complexing agent for the manufacture of a cleaning agent that contains at least one bleaching agent, and in particular for the manufacture of cleaning agent for fibers or hard surfaces, wherein said cleaning agent contains at least one peroxy compound. Another aspect of the present invention is a process for making at a cleaning agent by combining at least one inventive solid alkali metal salt (A) of an aminocarboxylate complexing agent with at least one bleaching agent, preferably at least one peroxy compound. Another aspect of the present invention is a cleaning agent, hereinafter also being referred to as inventive cleaning agent. Inventive cleaning agents contain at least one bleaching agent and at least one inventive solid alkali metal salt (A) of an aminocarboxylate complexing agent (e. g. powder). Inventive cleaning agents show a reduced tendency for yellowing and therefore have an extended shelve-life.

Examples of suitable peroxy compounds are sodium perborate, anhydrous or for example as monohydrate or as tetrahydrate or so-called dihydrate, sodium percarbonate, anhydrous or, for example, as monohydrate, hydrogen peroxide, persulfates, organic peracids such as peroxylauric acid, peroxystearic acid, peroxy-α-naphthoic acid, 1,12-diperoxydodecanedioic acid, perbenzoic acid, peroxylauric acid, 1,9-diperoxyazelaic acid, diperoxyisophthalic acid, in each case as free acid or as alkali metal salt, in particular as sodium salt, also sulfonylperoxy acids and cationic peroxy acids.

In a preferred embodiment, peroxy compound is selected from inorganic percarbonates, persulfates and perborates. Examples of sodium percarbonates are 2 Na₂CO₃·3 H₂O₂. Examples of sodium perborate are (Na₂[B(OH)₂(O₂)]₂), sometimes written as NaBO₂·O₂·3H₂O instead. Most preferred peroxy compound is sodium percarbonate

The term “cleaning agents” includes compositions for dishwashing, especially hand dishwash and automatic dishwashing and ware-washing, and compositions for hard surface cleaning such as, but not limited to compositions for bathroom cleaning, kitchen cleaning, floor cleaning, descaling of pipes, window cleaning, car cleaning including truck cleaning, furthermore, open plant cleaning, cleaning-in-place, metal cleaning, disinfectant cleaning, farm cleaning, high pressure cleaning, and in addition, laundry detergent compositions.

Such cleaning agents may be liquids, gels or preferably solids at ambient temperature, solids cleaning agents being preferred. They may be in the form of a powder or in the form of a unit dose, for example as a tablet or pouch.

In one embodiment of the present invention, inventive cleaning agents may contain
in the range of from 2 to 50% by weight of inventive solid alkali metal salt (A) of an aminocarboxylate complexing agent and
in the range of from 0.5 to 15% by weight of bleach.

Percentages are based on the solids content of the respective inventive cleaning agent.

Inventive solid alkali metal salts (A) of an aminocarboxylate complexing agent are excellently suited for the manufacture of laundry detergents or cleaners.

Inventive cleaning agents may contain further ingredients such as one or more surfactants that may be selected from non-ionic, zwitterionic, cationic, and anionic surfactants. Other ingredients that may be contained in inventive cleaning agents may be selected from bleach activators, bleach catalysts, corrosion inhibitors, sequestering agents other than chelating agent (A), enzymes, fragrances, dyestuffs, antifoams, and builders.

Particularly advantageous inventive cleaning agents may contain one or more complexing agents other than MGDA or GLDA. Advantageous detergent compositions for cleaners and advantageous laundry detergent compositions may contain one or more sequestrant (chelating agent) other than a mixture according to the present invention. Examples for sequestrants other than a mixture according to the present invention are IDS (iminodisuccinate), citrate, phosphonic acid derivatives, for example the disodium salt of hydroxyethane-1,1-diphosphonic acid (“HEDP”), and polymers with complexing groups like, for example, polyethyleneimine in which 20 to 90 mole-% of the N-atoms bear at least one CH₂COO⁻ group, and their respective alkali metal salts, especially their sodium salts, for example IDS-Na₄, and trisodium citrate, and phosphates such as STPP (sodium tripolyphosphate). Due to the fact that phosphates raise environmental concerns, it is preferred that advantageous inventive cleaning agents are free from phosphate. “Free from phosphate” should be understood in the context of the present invention, as meaning that the content of phosphate and polyphosphate is in sum in the range from 10 ppm to 0.2% by weight, determined by gravimetric methods and referring to the respective inventive cleaning agent.

Inventive cleaning agents may contain one or more surfactant, preferably one or more non-ionic surfactant.

Preferred non-ionic surfactants are alkoxylated alcohols, di- and multiblock copolymers of ethylene oxide and propylene oxide and reaction products of sorbitan with ethylene oxide or propylene oxide, alkyl polyglycosides (APG), hydroxyalkyl mixed ethers and amine oxides.

Preferred examples of alkoxylated alcohols and alkoxylated fatty alcohols are, for example, compounds of the general formula (II)

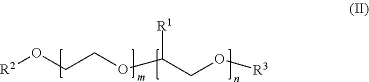

in which the variables are defined as follows:
R¹ is identical or different and selected from hydrogen and linear C₁-C₁₀-alkyl, preferably in each case identical and ethyl and particularly preferably hydrogen or methyl,
R² is selected from C₈-C₂₂-alkyl, branched or linear, for example n-C₈H₁₇, n-C₁₀H₂₁, n-C₁₂H₂₅, n-C₁₄H₂₉, n-C₁₆H₃₃ or n-C₁₈H₃₇,
R³ is selected from C₁-C₁₀-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl or isodecyl,
   m and n are in the range from zero to 300, where the sum of n and m is at least one, preferably in the range of from 3 to 50. Preferably, m is in the range from 1 to 100 and n is in the range from 0 to 30.

In one embodiment, compounds of the general formula (II) may be block copolymers or random copolymers, preference being given to block copolymers.

Other preferred examples of alkoxylated alcohols are, for example, compounds of the general formula (III)

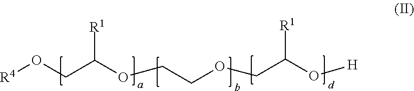

in which the variables are defined as follows:
R¹ is identical or different and selected from hydrogen and linear C₁-C₀-alkyl, preferably identical in each case and ethyl and particularly preferably hydrogen or methyl,
R⁴ is selected from C₆-C₂₀-alkyl, branched or linear, in particular n-C₈H₁₇, n-C₁₀H₂₁, n-C₁₂H₂₅, n-C₁₄H₂₉, n-C₁₆H₃₃, n-C₁₈H₃₇,
a is a number in the range from zero to 10, preferably from 1 to 6,
b is a number in the range from 1 to 80, preferably from 4 to 20,
d is a number in the range from zero to 50, preferably 4 to 25.

The sum a+b+d is preferably in the range of from 5 to 100, even more preferably in the range of from 9 to 50.

Preferred examples for hydroxyalkyl mixed ethers are compounds of the general formula (IV)

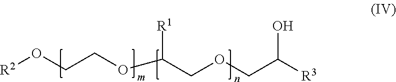

in which the variables are defined as follows:
R¹ is identical or different and selected from hydrogen and linear C₁-C₁₀-alkyl, preferably in each case identical and ethyl and particularly preferably hydrogen or methyl,
R² is selected from C₈-C₂₂-alkyl, branched or linear, for example iso-C₁₁H₂₃, iso-C₁₃H₂₇, n-C₈H₁₇, n-C₁₀H₂₁, n-C₁₂H₂₅, n-C₁₄H₂₉, n-C₁₆H₃₃ or n-C₁₈H₃₇,
R³ is selected from C₁-C₁₈-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl, n-dodecyl, n-tetradecyl, n-hexadecyl, and n-octadecyl.

The variables m and n are in the range from zero to 300, where the sum of n and m is at least one, preferably in the range of from 5 to 50. Preferably, m is in the range from 1 to 100 and n is in the range from 0 to 30.

Compounds of the general formula (II) and (Ill) may be block copolymers or random copolymers, preference being given to block copolymers.

Further suitable nonionic surfactants are selected from di- and multiblock copolymers, composed of ethylene oxide and propylene oxide. Further suitable nonionic surfactants are selected from ethoxylated or propoxylated sorbitan esters. Amine oxides or alkyl polyglycosides, especially linear C₄-C₁₆-alkyl polyglucosides and branched C₃-C₁₄-alkyl polyglycosides such as compounds of general average formula (V) are likewise suitable.

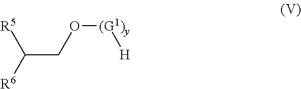

wherein the variables are defined as follows:
R⁵ is C₁-C₄-alkyl, in particular ethyl, n-propyl or isopropyl,
R⁶ is —(CH₂)₂—R⁵,
G¹ is selected from monosaccharides with 4 to 6 carbon atoms, especially from glucose and xylose,
y in the range of from 1.1 to 4, y being an average number.

Further examples of non-ionic surfactants are compounds of general formula (VII) and (VIII)

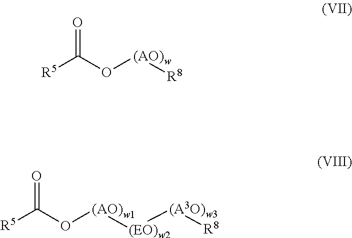

AO is selected from ethylene oxide, propylene oxide and butylene oxide,
EO is ethylene oxide, CH₂CH₂—O,
R³ selected from C₃-C₁₃-alkyl, branched or linear, and R⁵ is defined as above.
A³O is selected from propylene oxide and butylene oxide,
w is a number in the range of from 15 to 70, preferably 30 to 50,
w1 and w3 are numbers in the range of from 1 to 5, and
w2 is a number in the range of from 13 to 35.

An overview of suitable further nonionic surfactants can be found in EP-A 0 851 023 and in DE-A 198 19 187.

Mixtures of two or more different nonionic surfactants may also be present.

Other surfactants that may be present are selected from amphoteric (zwitterionic) surfactants and anionic surfactants and mixtures thereof.

Examples of amphoteric surfactants are those that bear a positive and a negative charge in the same molecule under use conditions. Preferred examples of amphoteric surfactants are so-called betaine-surfactants. Many examples of betaine-surfactants bear one quaternized nitrogen atom and one carboxylic acid group per molecule. A particularly preferred example of amphoteric surfactants is cocamidopropyl betaine (lauramidopropyl betaine).

Examples of amine oxide surfactants are compounds of the general formula (IX)

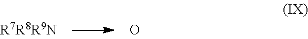

wherein R⁷, R³ and R⁹ are selected independently from each other from aliphatic, cycloaliphatic or C₂-C₄-alkylene C₁₀-C₂₀-alkylamido moieties. Preferably, R⁷ is selected from C₃-C₂₀-alkyl or C₂-C₄-alkylene C₁₀-C₂₀-alkylamido and R³ and R⁹ are both methyl.

A particularly preferred example is lauryl dimethyl aminoxide, sometimes also called lauramine oxide. A further particularly preferred example is cocamidylpropyl dimethylaminoxide, sometimes also called cocamidopropylamine oxide.

Examples of suitable anionic surfactants are alkali metal and ammonium salts of C₈-C₁₈-alkyl sulfates, of C₃-C₁₃-fatty alcohol polyether sulfates, of sulfuric acid half-esters of ethoxylated C₄-C₁₂-alkylphenols (ethoxylation: 1 to 50 mol of ethylene oxide/mol), C₁₂-C₁₈ sulfo fatty acid alkyl esters, for example of C₁₂-C₁₈ sulfo fatty acid methyl esters, furthermore of C₁₂-C₁₈-alkylsulfonic acids and of C₁₀-C₁₈-alkylarylsulfonic acids. Preference is given to the alkali metal salts of the aforementioned compounds, particularly preferably the sodium salts.

Further examples for suitable anionic surfactants are soaps, for example the sodium or potassium salts of stearoic acid, oleic acid, palmitic acid, ether carboxylates, and alkylether phosphates.

Preferably, laundry detergent compositions contain at least one anionic surfactant.

In one embodiment of the present invention, inventive cleaning agents that are determined to be used as laundry detergent compositions may contain 0.1 to 60% by weight of at least one surfactant, selected from anionic surfactants, amphoteric surfactants and amine oxide surfactants.

In one embodiment of the present invention, inventive cleaning agents that are determined to be used for hard surface cleaning may contain 0.1 to 60% by weight of at least one surfactant, selected from anionic surfactants, amphoteric surfactants and amine oxide surfactants.

In a preferred embodiment, inventive cleaning agents do not contain any anionic detergent.

Inventive cleaning agents may comprise one or more bleach catalysts. Bleach catalysts can be selected from bleach-boosting transition metal salts or transition metal complexes such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands and also cobalt-, iron-, copper- and ruthenium-amine complexes can also be used as bleach catalysts.

Inventive cleaning agents may comprise one or more bleach activators, for example N-methylmorpholinium-acetonitrile salts (“MMA salts”), trimethylammonium acetonitrile salts, N-acylimides such as, for example, N-nonanoylsuccinimide, 1,5-diacetyl-2,2-dioxohexahydro-1,3,5-triazine (“DADHT”) or nitrile quats (trimethylammonium acetonitrile salts).

Further examples of suitable bleach activators are tetraacetylethylenediamine (TAED) and tetraacetylhexylenediamine.

Inventive cleaning agents may comprise one or more corrosion inhibitors. In the present case, this is to be understood as including those compounds which inhibit the corrosion of metal. Examples of suitable corrosion inhibitors are triazoles, in particular benzotriazoles, bisbenzotriazoles, aminotriazoles, alkylaminotriazoles, also phenol derivatives such as, for example, hydroquinone, pyrocatechol, hydroxyhydroquinone, gallic acid, phloroglucinol or pyrogallol.

In one embodiment of the present invention, inventive cleaning agents comprise in total in the range from 0.1 to 1.5% by weight of corrosion inhibitor.

Inventive cleaning agents may comprise one or more builders, selected from organic and inorganic builders. Examples of suitable inorganic builders are sodium sulfate or sodium carbonate or silicates, in particular sodium disilicate and sodium metasilicate, zeolites, sheet silicates, in particular those of the formula α-Na₂Si₂O₅, β-Na₂Si₂O₅, and δ-Na₂Si₂O₅, also fatty acid sulfonates, α-hydroxypropionic acid, alkali metal malonates, fatty acid sulfonates, alkyl and alkenyl disuccinates, tartaric acid diacetate, tartaric acid monoacetate, oxidized starch, and polymeric builders, for example polycarboxylates and polyaspartic acid.

Examples of organic builders are especially polymers and copolymers other such as (co)polymers (B) and include polymers and copolymers than (co)polymer (B), or one additional (co)polymer (B). In one embodiment of the present invention, organic builders are selected from polycarboxylates, for example alkali metal salts of (meth)acrylic acid homopolymers or (meth)acrylic acid copolymers, partially or completely neutralized with alkali.

Suitable comonomers for (meth)acrylic acid are monoethylenically unsaturated dicarboxylic acids such as maleic acid, fumaric acid, maleic anhydride, itaconic acid and citraconic acid. A suitable polymer is in particular polyacrylic acid, which preferably has an average molecular weight M, in the range from 2000 to 40 000 g/mol, preferably 2000 to 10 000 g/mol, in particular 3000 to 8000 g/mol. Also of suitability are copolymeric polycarboxylates, in particular those of acrylic acid with methacrylic acid and of acrylic acid or methacrylic acid with maleic acid and/or fumaric acid, and in the same range of molecular weight.

It is also possible to use copolymers of at least one monomer from the group consisting of monoethylenically unsaturated C₃-C₁₀-mono- or C₄-C₁₀-dicarboxylic acids or anhydrides thereof, such as maleic acid, maleic anhydride, acrylic acid, methacrylic acid, fumaric acid, itaconic acid and citraconic acid, with at least one hydrophilic or hydrophobic monomer as listed below.

Suitable hydrophobic monomers are, for example, isobutene, diisobutene, butene, pentene, hexene and styrene, olefins with 10 or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C₂₂-α-olefin, a mixture of C₂₀-C₂₄-α-olefins and polyisobutene having on average 12 to 100 carbon atoms per molecule.

Suitable hydrophilic monomers are monomers with sulfonate or phosphonate groups, and also nonionic monomers with hydroxyl function or alkylene oxide groups. By way of example, mention may be made of: allyl alcohol, isoprenol, methoxypolyethylene glycol (meth)acrylate, methoxypolypropylene glycol (meth)acrylate, methoxypolybutylene glycol (meth)acrylate, methoxypoly(propylene oxide-co-ethylene oxide) (meth)acrylate, ethoxypolyethylene glycol (meth)acrylate, ethoxypolypropylene glycol (meth)acrylate, ethoxypolybutylene glycol (meth)acrylate and ethoxypoly(propylene oxide-co-ethylene oxide) (meth)acrylate. Polyalkylene glycols here may comprise 3 to 50, in particular 5 to 40 and especially 10 to 30 alkylene oxide units per molecule.

Particularly preferred sulfonic-acid-group-containing monomers here are 1-acrylamido-1-propanesulfonic acid, 2-acrylamido-2-propanesulfonic acid, 2-acrylamido-2-methylpropanesulfonic acid, 2-methacrylamido-2-methylpropanesulfonic acid, 3-methacrylamido-2-hydroxypropanesulfonic acid, allylsulfonic acid, methallylsulfonic acid, allyloxybenzenesulfonic acid, methallyloxybenzenesulfonic acid, 2-hydroxy-3-(2-propenyloxy)propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropyl methacrylate, sulfomethacrylamide, sulfomethylmethacrylamide, and salts of said acids, such as sodium, potassium or ammonium salts thereof.

Particularly preferred phosphonate-group-containing monomers are vinylphosphonic acid and its salts.

A further example of builders is carboxymethyl inulin.

Moreover, amphoteric polymers can also be used as builders.

Inventive cleaning agents may comprise, for example, in the range from in total 10 to 70% by weight, preferably from in total 10 to 50% by weight, more preferably up to 20% by weight, of builder.

In one embodiment of the present invention, inventive cleaning agents according to the invention may comprise one or more co-builders.

Inventive cleaning agents may comprise one or more antifoams, selected for example from silicone oils and paraffin oils.

In one embodiment of the present invention, inventive cleaning agents comprise in total in the range from 0.05 to 0.5% by weight of antifoam.

Inventive cleaning agents may comprise one or more enzymes. Examples of enzymes are lipases, hydrolases, amylases, proteases, cellulases, esterases, pectinases, lactases and peroxidases.

In one embodiment of the present invention, inventive cleaning agents may comprise, for example, up to 5% by weight of enzyme, preference being given to 0.1 to 3% by weight. Said enzyme may be stabilized, for example with the sodium salt of at least one C₁-C₃-carboxylic acid or C₄-C₁₀-dicarboxylic acid. Preferred are formates, acetates, adipates, and succinates.

In one embodiment of the present invention, inventive cleaning agents may comprise at least one zinc salt. Zinc salts can be selected from water-soluble and water-insoluble zinc salts. In this connection, within the context of the present invention, water-insoluble is used to refer to those zinc salts which, in distilled water at 25° C., have a solubility of 0.1 g/l or less. Zinc salts which have a higher solubility in water are accordingly referred to within the context of the present invention as water-soluble zinc salts.

In one embodiment of the present invention, zinc salt is selected from zinc benzoate, zinc gluconate, zinc lactate, zinc formate, ZnCl₂, ZnSO₄, zinc acetate, zinc citrate, Zn(NO₃)₂, Zn(CH₃SO₃)₂ and zinc gallate, preferably ZnCl₂, ZnSO₄, zinc acetate, zinc citrate, Zn(NO₃)₂, Zn(CH₃SO₃)₂ and zinc gallate.

In another embodiment of the present invention, zinc salt is selected from ZnO, ZnO·aq, Zn(OH)₂ and ZnCO₃. Preference is given to ZnO·aq.

In one embodiment of the present invention, zinc salt is selected from zinc oxides with an average particle diameter (weight-average) in the range from 10 nm to 100 μm.

The cation in zinc salt can be present in complexed form, for example complexed with ammonia ligands or water ligands, and in particular be present in hydrated form. To simplify the notation, within the context of the present invention, ligands are generally omitted if they are water ligands.

Depending on how the pH value of mixture according to the invention is adjusted, zinc salt can change. Thus, it is for example possible to use zinc acetate or ZnCl₂ for preparing formulation according to the invention, but this converts at a pH of 8 or 9 in an aqueous environment to ZnO, Zn(OH)₂ or ZnO·aq, which can be present in non-complexed or in complexed form.

Zinc salt may be present in those inventive cleaning agents that are solid at room temperature. In such inventive cleaning agents zinc salts are preferably present in the form of particles which have for example an average diameter (number-average) in the range from 10 nm to 100 μm, preferably 100 nm to 5 μm, determined for example by X-ray scattering.

Zinc salt may be present in those inventive cleaning agents that are liquid at room temperature. In such inventive cleaning agents zinc salts are preferably present in dissolved or in solid or in colloidal form.

In one embodiment of the present invention, inventive cleaning agents comprise in total in the range from 0.05 to 0.4% by weight of zinc salt, based in each case on the dry content of the cleaning agent in question.

Here, the fraction of zinc salt is given as zinc or zinc ions. From this, it is possible to calculate the counterion fraction.

In one embodiment of the present invention, inventive cleaning agents are free from heavy metals apart from zinc compounds. Within the context of the present, this may be understood as meaning that inventive cleaning agents are free from those heavy metal compounds which do not act as bleach catalysts, in particular of compounds of iron and of bismuth. Within the context of the present invention, “free from” in connection with heavy metal compounds is to be understood as meaning that the content of heavy metal compounds which do not act as bleach catalysts is in sum in the range from 0 to 100 ppm, determined by the leach method and based on the dry content. Preferably, inventive cleaning agents has, apart from zinc, a heavy metal content below 0.05 ppm, based on the dry content of the formulation in question. The fraction of zinc is thus not included.

Within the context of the present invention, “heavy metals” are deemed to be all metals with a specific density of at least 6 g/cm³ with the exception of zinc. In particular, the heavy metals are metals such as bismuth, iron, copper, lead, tin, nickel, cadmium and chromium.

Preferably, inventive cleaning agents comprise no measurable fractions of bismuth compounds, for example less than 1 ppm.

Inventive cleaning agents are excellent for cleaning hard surfaces and fibres. For example, they may be used in dishwashing applications, preferably automatic dishwashing applications.

In one embodiment of the present invention, inventive cleaning agents comprise one or more further ingredient such as fragrances, dyestuffs, organic solvents, buffers, disintegrants for tablets (“tabs”), and/or acids such as methylsulfonic acid.

From inventive solid compositions, e. g. granules or powders, exemplary detergent compositions for automatic dishwashing detergents can be formulated by mixing the respective components according to the following Table F.

TABLE F
Example detergent compositions for automatic dishwashing

| All amounts in g/sample | ADW.1 | ADW.2 | ADW.3 |
|---|---|---|---|
| Inventive solid particle | 30 | 22.5 | 15 |
| (solid alkali metal salt) | | | |
| Protease | 2.5 | 2.5 | 2.5 |
| Amylase | 1 | 1 | 1 |
| n-C₁₈H₃₇—O(CH₂CH₂O)₉H | 5 | 5 | 5 |
| Sodium percarbonate | 10.5 | 10.5 | 10.5 |
| TAED | 4 | 4 | 4 |
| Na₂CO₃ | 19.5 | 19.5 | 19.5 |
| Sodium citrate dihydrate | 15 | 22.5 | 30 |
| HEDP | 0.5 | 0.5 | 0.5 |
| ethoxylated polyethylenimine, | optionally: | optionally: | optionally: |
| 20 EO/NH group, Mₙ: 30,000 | 0.1 | 0.1 | 0.1 |
| g/mol | | | |

Laundry detergents according to the invention are useful for laundering any type of laundry, and any type of fibres. Fibres can be of natural or synthetic origin, or they can be mixtures of natural of natural and synthetic fibres. Examples of fibers of natural origin are cotton and wool. Examples for fibers of synthetic origin are polyurethane fibers such as Spandex® or Lycra®, polyester fibers, or polyamide fibers. Fibers may be single fibers or parts of textiles such as knitwear, wovens, or nonwovens.

Another aspect of the present invention is a process for making tablets for automatic dishwashing from an inventive solid alkali metal salt (A) of an aminocarboxylate complexing agent, e. g. a powder or granule, wherein said granule or powder is selected from inventive granules and inventive powders, respectively. Said process is hereinafter also referred to as pelletizing process according to the invention.

Inventive tablets are preferably made with the help of a machine, for example a tablet press.

The pelletizing process according to the invention can be carried out by mixing an inventive solid alkali metal salt (A) of an aminocarboxylate complexing agent, e. g. powder, with at least one non-ionic surfactant and optionally one or more further substance and then compressing the mixture to give tablets. Examples of suitable non-ionic surfactants and further substances such as builders, enzymes are listed above. Particularly preferred examples of non-ionic surfactants are hydroxy mixed ethers, for example hydroxy mixed ethers of the general formula (V) Some of the aspects of the present invention are illustrated by the following working examples.

### PREPARATION OF SAMPLES

### Comparative Example

Trilon® M (MGDA) solution was dried in an agglomerating spray drying process, using a commercially available apparatus from the vendor Anhydo called SBD (spray bed dryer).

This SBD apparatus is a spray tower with an integrated fluidized bed. An amount of 4 kg spray powder was introduced as initial filling. An amount of 350 Nm³/h nitrogen (drying gas) was introduced with a temperature of 200-210° C. into the top of the tower around the two-component-nozzle. A feed of 25 kg/h of Trilon® M liquid (as is) was atomized by this two-component-nozzle und sprayed into the dust cloud, that is in the dryer. The exhaust air, leaving the dryer through the top/side outlets, carried the dust (i. e. fine particles, with an average diameter of at most 300 μm) out of the dryer. The dust is separated from the exhaust air by a cyclone and is reintroduced around the nozzle. The liquid is sprayed in a way, that it glued the dust particles together, so they agglomerate. New dust is generated by overspray (droplets do not hit dust particles and become dried). Agglomerates, that are heavy enough are moved by gravity into the internal fluidized bed and become pre-dried by the fluidization air of 80 Nm³/h and about 62° C. A weir and a discharge valve keep a dedicated filling level of the internal fluidized bed of 4-8 mbar differential pressure. Product that left the dryer is collected as targeted agglomerates.

Trilon® M is commercially available from BASF SE and refers to MGDA chelating agent.

### Inventive Example 1

A Slurry was prepared by mixing 66.2 kg of Trilon® M liquid, 32.8 kg of Trilon® M granules and 1 kg of seed material. The slurry was mixed and tempered at about 70° C. for 3 h.

Trilon® M solution was dried in an agglomerating spray drying process, using a commercially available apparatus from the vendor Anhydo called SBD (spray bed dryer).

This SBD apparatus is a spray tower with an integrated fluidized bed. An amount of 4 kg spray powder was introduced as initial filling. An amount of 325-340 Nm³/h nitrogen (drying gas) was introduced with a temperature of 200-210° C. into the top of the tower around the two-component-nozzle. A feed of 45-50 kg/h of the said Trilon M® slurry was atomized by this two-component-nozzle und sprayed into the dust cloud, that is in the dryer. The exhaust air, leaving the dryer through the top/side outlets, carried the dust (i. e. fine particles, with an average diameter of at most 300 μm) out of the dryer. The dust is separated from the exhaust air by a cyclone and is reintroduced around the nozzle. The liquid is sprayed in a way, that it clued the dust particles together, so they agglomerate. New dust is generated by overspray (droplets do not hit dust particles and become dried). Agglomerates, that are heavy enough are moved by gravity into the internal fluidized bed and become pre-dried by the fluidization air of 100 Nm³/h and about 65-75° C. A weir and a discharge valve keep a dedicated filling level of the internal fluidized bed of 5-10 mbar differential pressure. Product, that leaved the dryer is collected as targeted agglomerates.

### Inventive Example 2

A Slurry was prepared by mixing 34.79 kg water, 3.23 kg Sokalan® CP50 granules, 60.9 kg Trilon® M granules and 1.0 kg crystal form II seeds. The slurry was mixed and tempered at about 70° C. for 3 h.

Then the agglomerating spray drying process was used to process the so obtained slurry, like it is described in the first inventive example.

Sokalan® CP50 is commercially available from BASF SE and refers to a modified polycarboxylate polymer (sodium salt).

### Application Tests

### Percarbonate Stability

The MGDA samples (10 g of MGDA sample, prepared as described above, together with 5 g of percarbonate) were stored in a cell culture flask with a membrane in the cap at 35° C. under 70% relative humidity in the climate cabinet.

The measurement of the b value was done with a Mach 5 apparatus.

| | | | b-value | | |
|---|---|---|---|---|---|
| | Zero value | 1 week | 2 weeks | 3 weeks | 4 weeks |
| Comparative Example | 3.90 | 4.72 | 11.57 | 11.01 | 9.86 |
| Inventive Example 1 | 2.58 | 3.50 | 6.53 | 9.76 | 9.35 |
| Inventive Example 2 | 3.32 | 6.41 | 9.22 | 9.92 | 13.03 |

### Hygroscopicity

The MGDA samples (5 g of sample, prepared as described above) were stored in a Petri dish at 35° C. under 70% relative humidity in a climate chamber (incubator).

| | | | Comparative | Inventive | Inventive |
|---|---|---|---|---|---|
| | | | Example | example 1 | example 2 |
| Water uptake (%): | | | 0 | 0 | 0 |
| after | 1 | h | 8.2 | 6.8 | 6.0 |
| after | 2 | h | 12.3 | 9.7 | 9.0 |
| after | 4 | h | 16.6 | 12.8 | 11.6 |
| after | 6 | h | 19.3 | 14.5 | 13.4 |
| after | 8 | h | 21.3 | 15.5 | 14.7 |
| after | 24 | h | 29.4 | 16.2 | 18.0 |
| after | 48 | h | 33.6 | 16.5 | 15.6 |
| after | 168 | h | 29.0 | 15.3 | 14.1 |

The experimental data show, inter alia, that starting from a slurry in an agglomerative spray-drying process leads to surprisingly improved results in the product, as compared to the use of a solution. For example, the take-up of water in the resulting, inventive particles is considerably reduced, and the resulting inventive particles show a considerably lower tendency for coloration, in the presence of peroxy compounds.

1. A process for manufacturing solid particles comprising at least one aminocarboxylate complexing agent or its salts with alkali metals, comprising spray-drying a slurry comprising aminocarboxylate complexing agent, in the presence of fine particles with an average diameter of at most 300 μm, under agglomerating conditions.

2. The process according to claim 1, wherein the slurry comprises at least 45% by weight of aminocarboxylate complexing agent and optionally seed particles of solid aminocarboxylate complexing agent.

3. The process according to claim 1, wherein the aminocarboxylate complexing agent is selected from MGDA, GLDA, EDDS, or IDS.

4. The process according to claim 1, wherein the process comprises the steps of
(a) forming an aqueous slurry, comprising seed particles of solid aminocarboxylate complexing agent, optionally at least 45% by weight of solid aminocarboxylate complexing agent, relative to the total weight of the aqueous slurry,
(b) spray drying the slurry in a spray drying apparatus, equipped with a fluidized bed,
wherein particles P1 with a particle diameter which is less than the targeted particle diameter P leave the spray drying apparatus through an exhaust air outlet on the head of the dryer, are removed by a cyclone and re-introduced into the drying apparatus, and wherein particles P1 agglomerate in the spray drying apparatus when they are re-introduced into the spray-drying apparatus to form particles P2 with an increased average particle diameter and/or weight, as compared to particles P1, and wherein particles P2, when they have the targeted particle diameter P, fall into the fluidized bed and are, optionally, air-classified and, optionally, post-dried, to receive free-flowing particles P3.

5. The process according to claim 4, wherein particles P3 are compacted into compacted aggregates.

6. The process according to claim 4, wherein the slurry provided in step (a) contains at least 0.1 weight % of a further organic or inorganic compound or mixtures thereof.

7. The process according to claim 4, wherein the slurry provided in step (a) contains at least 0.1 weight % of a (co)polymer (B) selected from (co)polymers of (meth)acrylic acid and polyethyleneimines, non-substituted or substituted with alkoxy groups or CH₂COOH groups that may be neutralized with alkali metal, and/or polyaspartates and/or polyepoxysuccinic acid.

8. The process according to claim 4, wherein the slurry is tempered in step (a) before entering the spray-drying apparatus in step (b).

9. The process according to claim 1, wherein the process is performed in an agglomerative spray-drying apparatus.

10. The process according to claim 1, wherein the process is performed in an agglomerative spray-drying apparatus with a height of at least 5 meters.

11. The process according to claim 1, wherein the resulting particle is matured.

12. The process according to claim 4, wherein the slurry formed in step (a) is heated to a temperature of at least 70° C. for a period of from 10 minutes to 8 hours, to increase the content of orthorhombic aminocarboxylate complexing agent in the slurry.

13. A solid particle, comprising at least one aminocarboxylate complexing agent, obtainable or obtained by the process according to claim 1.

14. The solid particle according to claim 13, with a particle size of 50 to 1400 μm, and/or wherein the particle has a crystallinity of at least 10%, as determined by X-ray diffraction, and/or a content of orthorhombic crystal form of at least 30%, as determined by X-ray diffraction.

15. The solid particle according to claim 13, with a moisture content of not more than 30 wt. %.

16. A cleaning agent comprising a solid particle according to claim 13.

17. The cleaning agent according to claim 16, further comprising an antimicrobial agent selected from 2-phenoxyethanol.

18. The process according to claim 1, wherein the solid particles are free-flowing solid particles.

19. The cleaning agent according to claim 17, wherein the phenoxyethanol is in an amount of 0.1 to 2% by weight of the cleaning agent.
